(19) United States
(12) Patent Application Publication
(10) Pub. No.: US 2012/0263658 A1
(43) Pub. Date: Oct. 18, 2012

(54) FLAVORED CHEWABLE FOAMS AND A PROCESS FOR THEIR PRODUCTION

(75) Inventors: Steffen HOFACKER, Odenthal (DE); Thomas FELLER, Solingen (DE); Thomas MICHAELIS, Leverkusen (DE); Sebastian DOERR, Duesseldorf (DE); Dirk SCHWANNECKE, Murray Hill, NJ (US); Meike NIESTEN, Koln (DE); Thorsten RISCHE, Unna (DE)

(73) Assignee: Bayer MaterialScience AG, Leverkusen (DE)

(21) Appl. No.: 13/489,704

(22) Filed: Jun. 6, 2012

Related U.S. Application Data
(63) Continuation of application No. 12/913,445, filed on Oct. 27, 2010, now abandoned.
Application No. 12/913,445 is a division of application No. 11/586,099, filed on Oct. 25, 2006, now abandoned.

(51) Int. Cl. A61K 8/87; A61Q 11/00

(52) U.S. Cl. 424/54

(57) ABSTRACT

Flavored chewable foams for the oral care sector are produced from a polyurethane-polyurea and a sensorially active substance (e.g., taste substance and/or aroma substance) by foaming a polyurethane-urea dispersion, drying that dispersion and incorporating the sensorially active substance into the polyurethane-urea dispersion before, during or after foaming of the dispersion.

### RELATED APPLICATIONS

This application is a Continuation of U.S. Ser. No. 12/913,445, which was filed Oct. 27, 2010, which is a Divisional of U.S. application Ser. No. 11/586,099, now abandon, filed Oct. 25, 2006, each of which is incorporated herein by reference in their entireties.

### BACKGROUND OF THE INVENTION

The invention relates to flavored chewable foams for the oral care sector made with polyurethane-polyureas and taste substances and/or aroma substances and to a method for production thereof.

Organic polymers are frequently used as raw materials in cosmetic products. They may be found in cosmetic products such as hair sprays, hair-gels, mascara, lipsticks, creams etc. In the oral care sector, polymers have been used, for example, in toothbrushes, dental flosses, etc.

In view of the increasing need for oral care during periods between meals or after consumption of a snack, sweets, nicotine, alcohol, etc.) and increased mobility (for example, during air or train travel) in which conventional teeth cleaning with water, toothpaste and toothbrush is not possible, products such as dental care chewing gums or else dental care wipes have been developed.

Dental care chewing gums are essentially composed of gum base. The gum base is generally a natural or synthetic polymer such as latex, polyvinyl ethers, polyisobutylene vinyl ethers, polyisobutene, etc. Such dental care chewing gums, as dental care compositions, generally contain pH-controlling substances which counteract the development of tooth decay (caries). However, because of their plastic behavior, such dental care chewing gums scarcely contribute to cleaning the chewing surfaces or tooth sides. In addition, chewing gums generally have the disadvantage that they must frequently be mechanically removed from public streets and spaces, and disposed of, which, in view of their adhesive properties, can lead to considerable cleaning expenditure.

Teeth wipes (for example Oral-B Brush Aways™, Gillette GmbH & Co. OHG, Germany) are distinguished in that they achieve good cleaning action of the tooth sides by applying the teeth wipe onto a finger and by rubbing the teeth. However, the mode of employing such teeth cleaning wipes in public has gained little acceptance for aesthetic reasons and is thus not an accepted alternative to using a conventional toothbrush.

DE 102006019742.9 describes novel chewable foams based on polyurethane-polyureas.

### SUMMARY OF THE INVENTION

It has now been found that polyurethane-polyurea chewable foams can readily be used for oral care when they are flavored.

### DETAILED DESCRIPTION OF THE INVENTION

The invention relates to flavored chewable foams based on polyurethane-polyureas.

Particularly advantageous chewable foams are those which have a 100% modulus of from 0.4 to 5.0 MPa, at a tensile strength of 1 to 55 MPa and an extensibility of 300 to 1800%.

The present invention further relates to a process for producing such flavored chewable foams in which one or more polyurethane-polyurea dispersions (I), if appropriate together with further components of the chewable foams, are foamed and subsequently dried. Flavoring compositions (II) may be added before, during or after production of the chewable foam.

Preference is given to addition of the flavoring compositions as soon as before, and/or after, foaming.

Such polyurethane-polyurea dispersions (I) are obtainable by producing

A) isocyanate-functional prepolymers from
a1) aliphatic or cycloaliphatic polyisocyanates,
a2) polymeric polyols having number-average molecular weights of 400 to 8000 g/mol and OH functionalities of 1.5 to 6,
a3) if appropriate hydroxy functional, ionic or potentially ionic and/or nonionic hydrophilizing agents,

B) their free NCO groups then being in whole or in part reacted with
b1) amino functional compounds having molecular weights of 32 to 400 g/mol and/or
b2) amino functional, ionic-or potentially ionic hydrophilizing agents

with chain extension and the prepolymers being dispersed in water before, during or after step B), if appropriate potentially ionic groups present being able to, be converted into the ionic form by partial or complete reaction with a neutralizing agent.

Isocyanate-reactive groups are, for example, amino, hydroxyl or thiol groups.

In a1), use is typically made of 1,6-hexamethylene diisocyanate, isophorone diisocyanate, the isomeric bis(4,4′-isocyanatocyclohexyl)methanes and also mixtures thereof. Isocyanate-reactive groups include, for example, amino, hydroxyl and thiol groups.

In a1), use is typically made of 1,6-hexamethylene diisocyanate, isophorone diisocyanate, the isomeric bis(4,4′-isocyanatocyclohexyl)methanes and also mixtures thereof.

It is likewise possible to use modified diisocyanates having uretdione, isocyanurate, urethane, allophanate, biuret, iminooxadiazinedione and/or oxadiazinetrione structure, and also the nonmodified polyisocyanates having more than 2 NCO groups per molecule such as 4-isocyanatomethyl-1,8-octane diisocyanate (nonane triisocyanate) or triphenylmethane 4,4′,4″-triisocyanate.

Particularly preferably, the compounds of the component a1) are polyisocyanates or polyisocyanate mixtures of the abovementioned type solely containing aliphatically and/or cycloaliphatically bound, isocyanate groups and a mean NCO functionality of the mixture of 2 to 4, preferably 2 to 2.6, and particularly preferably 2 to 2.4.

As components in a2), use is made of polymeric polyols having number-average molecular weights of 400 to 6000 g/mol, particularly preferably 600 to 3000 g/mol. These preferably have OH functionalities of 1.8 to 3, particularly preferably 1.9 to 2.1.

Aforesaid polymeric polyols which are known per se in polyurethane coating technology are polyester polyols, polycarbonate polyols, polyether polyols, polyester polycarbonate polyols and polyether carbonate polyols. These can be used in a2) individually or in any desired mixtures with one another.

The polymeric polyols used of the abovementioned type are preferably those having an aliphatic backbone. Preferably, use is made of aliphatic polycarbonate polyols, polyether polyols or any desired mixtures thereof.

Preferred embodiments of the polyurethane dispersions (I) contain, as component a2), a mixture of polycarbonate polyols and polytetramethylene glycol polyols, the fraction in the mixture being 35 to 70% by weight of polytetramethylene glycol polyols and 30 to 65% by weight of polycarbonate polyols, with the provision that the total of the percentages by weight of polycarbonate and polytetramethylene glycol polyols is 100% by weight.

Hydroxy functional, ionic or potentially ionic hydrophilizing agents a3) are taken to mean all compounds which have at least one isocyanate-reactive hydroxyl group and also at least one functionality such as for example —COOY, —SO₃Y, —PO(OY)₂ (Y⁺ for example=H⁺, NH₄⁺, metal cation), —NR₂, —NR₃⁺ (R═H, alkyl, aryl) which, on interaction with aqueous media, enters into a pH-dependent dissociation equilibrium and in this manner can be negatively, positively or neutrally charged.

Suitable ionically or potentially ionically hydrophilizing compounds corresponding to the definition of component a3) are, for example, mono- and dihydroxycarboxylic acids, mono- and dihydroxysulfonic acids, and also mono- and dihydroxyphosphonic acids and salts thereof such as dimethylolpropionic acid, dimethylolbutyric acid, hydroxypivalic acid, malic acid, citric acid, glycolic acid, lactic acid, the propoxylated adduct of 2-butenediol and NaHSO₃, e.g. described in DE-A 2 446 440 (pages 5-9, formulae I-III) and also compounds which contain, as hydrophilic structural components, for example amine-based building blocks such as N-methyldiethanolamine which are convertible into cationic groups.

Preferred ionic or potentially ionic hydrophilizing agents of the component a3) are such of the abovementioned type which are anionically hydrophilizing, preferably via carboxyl or carboxylate and/or sulfonate groups.

Particularly preferred ionically or potentially ionically hydrophilizing agents are those which contain carboxyl and/or sulfonate groups as anionic or potentially anionic groups, such as the salts of dimethylolpropionic acid or dimethylolbutyric acid.

Suitable nonionically hydrophilizing compounds of component a3) are, for example, polyoxyalkylene ethers which contain at least one hydroxyl group as isocyanate-reactive group.

Examples are the monohydroxy functional polyalkylene oxide polyether alcohols having a statistical mean of 5 to 70, preferably 7 to 55, ethylene oxide units per molecule, such as are accessible in a manner known per se by alkoxylating suitable starter molecules (e.g. in Ullmanns Encyclopädie der technischen Chemie [Ullmann's Encyclopedia of Industrial Chemistry], 4th edition, volume 19, Verlag Chemie, Weinheim, pages 31-38).

These are either pure polyethylene oxide ethers or mixed polyalkylene oxide ethers, with them containing at, least 30 mol %, preferably at least 40 mol %, ethylene oxide units, based on all alkylene oxide units present.

Particularly preferred nonionic compounds arc monofunctional mixed polyalkylene oxide polyethers which have 40 to 100 mol % ethylene oxide and 0 to 60 mol % propylene oxide units.

Suitable starter molecules for such nonionic hydrophilizing agents include: saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane, and tetrahydrofurfuryl alcohol; diethylene glycol monoalkyl ethers, such as diethylene glycol monobutyl ether; unsaturated alcohols such as allyl alcohol, 1,1-dimethylallyl alcohol or oleyl alcohol; aromatic alcohols such as phenol, the isomeric cresols and methoxyphenols; araliphatic alcohols such as benzyl alcohol, anisyl alcohol and cinnamyl alcohol; secondary monoamines such as dimethylamine, diethylamine, dipropylamine, diisopropylamine, dibutylamine, bis(2-ethylhexyl)amine, N-methyl- and N-ethylcyclohexylamine and dicyclohexylamine; and also heterocyclic secondary amines such as morpholine, pyrrolidine, piperidine and 1H-pyrazole. Preferred starter molecules are saturated monoalcohols of the abovementioned type. Particularly preferred starter molecules are diethylene glycol monobutyl ether and n-butanol.

Alkylene oxides suitable for the alkoxylation reaction are, in particular, ethylene oxide and propylene oxide, which can be used in the alkoxylation reaction in any desired sequence or else in a mixture.

In component b1), use can be made of di- or polyamines such as 1,2-ethylenediamine, 1,2- and 1,3-diaminopropane, 1,4-diaminobutane, 1,6-diaminohexane, isophoronediamine, mixtures of isomers of 2,2,4- and 2,4,4-trimethylhexamethylenediamine, 2-methylpentamethylenediamine, diethylenetriamine, and 4,4-diaminodicyclohexylmethane and/or dimethylethylenediamine.

In addition, as component b1), use can also be made of compounds which, in addition to a primary amino group, also have secondary amino groups or, in addition to an amino group (primary or secondary), also have OH groups. Examples of these are primary/secondary amines such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

In addition, as component b1), use can also be made of monofunctional amine compounds such as, for example, methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine and suitable substituted derivatives thereof, amidoamines of diprimary amines and monocarboxylic acids, monoketimines of diprimary amines, primary/tertiary amines such as N,N-dimethylaminopropylamine.

Preferably, use is made of 1,2-ethylenediamine, 1,4-diaminobutane, isophoronediamine and diethylenetriamine.

Ionically or potentially ionically hydrophilizing compounds of the component b2) are taken to mean all compounds which have at least one isocyanate-reactive amino group and also at least one functionality such as, for example, —COOY, —SO₃Y, —PO(OY)₂ (Y for example=H, NH₄⁺, metal cation), which, on interaction with aqueous media, enter into a pH-dependent dissociation equilibrium and in this manner can be positively, negatively or neutrally charged.

Suitable ionically or potentially ionically hydrophilizing compounds are, for example, mono- and diaminocarboxylic acids, mono- and diaminosulfonic acids and also mono- and diaminophosphonic acids and salts thereof. Examples of such ionic or potentially ionic hydrophilizing agents are N-(2-aminoethyl)-β-alanine, 2-(2-aminoethylamino)ethanesulfonic acid, ethylenediaminepropylsulfonic or butylsulfonic acid, 1,2- or 1,3-propylenediamine-β-ethylsulfonic acid, glycine, alanine, taurine, lysine, 3,5-diaminobenzoic acid and the addition product of IPDI and acrylic acid (EP-A 0 916 647, example 1). In addition, use can be made of cyclohexylaminopropanesulfonic acid (CAPS) from WO-A 01/88006 as anionic or potentially anionic hydrophilizing agent.

Preferred ionic or potentially ionic hydrophilizing agents b2) are those which contain carboxyl and/or sulfonate groups as anionic or potentially anionic groups, such as the salts of N-(2-aminoethyl)-β-alanine, 2-(2-aminoethylamino)ethanesulfonic acid or the addition product of IPDI and acrylic acid (EP-A 0 916 647, example 1).

For the hydrophilization, preferably use is made of a mixture of anionic or potentially anionic hydrophilizing agents and nonionic hydrophilizing agents.

The ratio of NCO groups of the compounds of component a 1) to NCO-reactive groups of the components a2) to a3) in production of the NCO-functional prepolymer is 1.2 to 3.0, preferably 1.3 to 2.5.

The amino functional compounds B) are used in an amount such that the equivalent ratio of isocyanate-reactive amino groups of these compounds to the free isocyanate groups of the prepolymer is 50 to 125%, preferably 60 to 120%.

In a preferred embodiment, use is made of anionically and nonionically hydrophilized polyurethane dispersions, for their production use being made of the components a1) to a3) and b1) to b2) in the following amounts, the individual amounts totaling 100% by weight:

10 to 30% by weight of component a1),

65 to 85% by weight of a2),

0.5 to 14% by weight sum of component b1)

0.1 to 13.5% by weight sum of components a3) and b2), based on the total amounts of components a1) to a3), use being made of 0.5 to 3.0% by weight of anionic or potentially anionic hydrophilizing agents.

Particularly preferred embodiments of the polyurethane dispersions (I), as component a1), contain isophorone diisocyanate and/or 1,6-hexamethylene diisocyanate and/or the isomeric bis(4,4′-isocyanatocyclohexyl)methanes in combination with a2) a mixture of polycarbonate polyols and polytetramethylene glycol polyols.

The respective fraction of polymeric polyols in mixture a2) is 35 to 70% by weight of polytetramethylene glycol polyols and 30 to 65% by weight of polycarbonate polyols, in each case with the proviso that the total of the percentage by weight of the polycarbonate and polytetramethylene glycol polyols is 100% by weight.

Such polyurethane dispersions can be produced in one or more stage(s) in homogeneous or multistage reaction, partially in disperse phase. After polyaddition, complete or carried out in part, of a1) to a3), a dispersion, emulsification or solution step proceeds. Subsequently, if appropriate, further polyaddition or modification in disperse phase proceeds.

All methods known from the prior art can be used here such as, for example, prepolymer mixing methods, acetone methods or melt dispersion methods. Preferably, the process proceeds via the acetone method.

For preparation according to the acetone method, customarily components a2) and a3) which must not have any primary or secondary amino groups, and the polyisocyanate component a1), for production of an isocyanate-functional polyurethane prepolymer, are charged in whole or in part and if appropriate diluted with a solvent which is water-miscible but inert to isocyanate groups, and heated to temperatures in the range from 50 to 120° C. To accelerate the isocyanate addition reaction, the catalysts known in polyurethane chemistry can be added.

Suitable solvents are, the customary aliphatic, ketofunctional solvents such as acetone, 2-butanone, which can be added not only at the start of production, but also, if appropriate, in parts later. Preference is given to acetone and 2-butanone.

Subsequently, any components a1) to a3) which are not yet added at the start of the reaction are added.

The reaction of components a1) to a3) to form the prepolymer proceeds partially or completely, but preferably completely. In such a manner polyurethane prepolymers which contain free isocyanate groups are obtained in the absence of solvent or in solution.

Subsequently, in a further method step, if this is not yet proceeded, or only in part, the resultant prepolymer is dissolved using aliphatic ketones such as acetone or 2-butanone.

The amine components b1), b2) can if appropriate be used individually or in mixtures in water- or solvent-diluted form in the inventive method, in principle any sequence of addition being possible.

If water or organic solvents are used in conjunction as diluents, the diluent content in the component used in B) for chain extension is preferably 30 to 95% by weight.

Dispersion preferably proceeds subsequently to chain extension. For this the dissolved and chain-lengthened polyurethane polymer, if appropriate under severe shear, for example vigorous stirring, is either charged into the dispersion water, or, vice versa, the dispersion water is stirred into the chain-lengthened polyurethane polymer solutions. Preferably, the water is added to the dissolved chain-lengthened polyurethane polymer.

The solvent still present in the dispersions after the dispersion step is customarily subsequently removed by distillation. It is also possible for removal to proceed as soon as during dispersion.

The residual content of organic solvents in the dispersions essential to the invention is typically less than 1.0% by weight, preferably less than 0.3% by weight, based on the total dispersion.

The pH of the dispersions essential to the invention is typically less than 9.0, preferably less than 8.0.

The solids content of the polyurethane dispersion is typically 40 to 63% by weight.

In the production of the inventive flavored chewable foams, in addition to the dispersions (I) and flavoring compositions (II), use can also be made in conjunction of foam agents/stabilizers (III), thickeners (IV), and cosmetic additives (V).

Flavoring compositions (II) within the meaning of the present invention contain sensorially active substances, which can be volatile. (aroma substances) or nonvolatile (taste substances). These compositions (II) are incorporated into the inventive chewable foams in amounts such that a sensory action occurs when the bases are chewed.

The (volatile) aroma substances can be perceived by humans both orthonasally and retronasally. The taste substances interact with the taste receptors of the tongue and are responsible for the gustatory (taste) impressions sweet, sour, bitter, salty and umami, in addition, other frequently trigeminal stimuli are perceived, such as pungent, burning, cooling, electrifying (“tingling”) or prickling effects.

Customarily, the sensorially active substances include at least one aroma substance, preferably 2, 3, 4, 5, 6, 7, 8, 9, 10 or more.

Taste substances within the meaning of the present invention thus comprise, inter alia, (mucosa-) cooling agents, (mucosa)-heating agents, pungent-tasting substances, sweeteners, organic or inorganic acidulants such as malic acid, acetic acid, citric acid, tartaric acid and/or phosphoric acid, bitter substances such as quinine, caffeine, limonin, amarogentin, humolones, lupolones, catechins and/or tannins, and also edible mineral salts such as sodium chloride, potassium chloride, magnesium chloride and/or sodium phosphates.

Advantageous aroma substances which are suitable as component of the inventive flavored chewable foams are found, for example, in S. Arctander, Perfume and Flavor Chemicals, Vol. I und II, Montclair, N. J. 1969, in-house publisher, or K. Bauer, D. Garbe and H. Surburg, Common Fragrance and Flavor Materials, 4th Edition, Wiley-VCH, Weinheim 2001.

Those which may be mentioned by way of example are aliphatic saturated or unsaturated esters such as ethyl butyrate or allyl caproate; aromatic esters such as benzyl acetate or methyl salicylate; cyclic alcohols such as menthol; aliphatic alcohols such as isoamyl alcohol or 3-octanol; aromatic alcohols such as benzyl alcohol; aliphatic saturated or unsaturated aldehydes such as acetaldehyde or isobutyraldehyde; aromatic aldehydes such as benzaldehyde or vanillin; ketones such as menthone, carvone; cyclic ethers such as 4-hydroxy-5-methylfuranone; aromatic ethers such as p-methoxybenzaldehyde or guaiacol; lactones such as gamma-decalactone; terpenes such as limonene, linalool, terpinene, terpineol or citral.

Preferred aroma substances are selected from the group consisting of menthol (preferably 1-menthol and/or racemic menthol), anethole, anisole, anisaldehyde, anisyl alcohol, (racemic) neomenthol, eucalyptol (1,8-cineol), menthone (preferably L-menthone), isomenthone (preferably D-isomenthone), isopulegol, menthyl acetate (preferably L-menthyl acetate), menthyl propionate, carvone (preferably (−)-carvone, if appropriate as component of a spearmint oil), methyl salicylate (if appropriate as component of a wintergreen oil), eugenol acetate, isoeugenol methyl ether, beta-homocyclocitral, eugenol, isobutyraldehyde, 3-octanol, dimethyl sulfide, trans-2-hexenal, cis-3-hexenol, 4-terpineol, piperitone, linalool, 8-ocimenyl acetate, isoamyl alcohol, isovaleraldehyde, alpha-pinene, beta-pinene, limonene (preferably D-limonene, if appropriate as component of an essential oil), piperitone, trans-sabinene hydrate, menthofuran, caryophyllene, germacrene D, cinnamaldehyde, mintlactone, thymol, gamma-octalactone, gamma-nonalactone, gamma-decalactone, (1,3E,5Z)-undecatriene, 2-butanone, ethyl formate, 3-octyl acetate, isoamyl isovalerate, cis- and trans-carvyl acetate, p-cymene, damascenone, damascone, cis-rose oxide, trans-rose oxide, fenchol, acetaldehyde diethyl acetal, 1-ethoxyethyl acetate, cis-4-heptenal, cis-jasmone, methyl dihydrojasmonate, menthyl methyl ether, myrtenyl acetate, 2-phenylethyl alcohol, 2-phenylethyl isobutyrate, 2-phenylethyl isovalerate, geraniol and nerol.

Other preferred aroma substances are essential oils and extracts, tinctures and balsams, such as anise oil, basil oil, bergamot oil, bitter almond oil, camphor oil, citronella oil, citron oil; eucalyptus citriodora oil, eucalyptus oil, fennel oil, grapefruit oil, ginger oil, camomile oil, spearmint oil, cumin oil, limetta oil, mandarin oil, muscat oil (in particular muscat blossom oil=mace oil), myrrh oil, clove oil, clove buds, orange oil, oregano oil, parsley (seed) oil, peppermint oil, rosemary oil, sage oil (muscatel sage, Dalmation or Spanish sage oil), star anise oil, thyme oil, vanilla extract, juniper oil (in particular juniper berry oil), wintergreen oil, cinnamon leaf oil; cinnamon bark oil, and also fractions thereof, and components isolated therefrom.

To achieve a refreshing action in the oral, throat and/or nasal cavity, preference is given to aroma substances from the group consisting of 1-menthol, racemic menthol, anethole, anisaldehyde, anisyl alcohol, neomenthol, eucalyptol (1,8-cineol), L-menthone, D-isomenthone, isopulegol, L-menthyl acetate, (−)-carvone, methyl salicylate, trans-2-hexenal, cis-3-hexenol, 4-terpineol, linalool, 8-ocimenyl acetate, alpha-pinene, D-limonene, (+)-menthofuran, cinnamaldehyde and menthyl methyl ether.

Particularly preferred substances with a refreshing action in the oral, throat and/or nasal cavity are menthol, menthone, isomenthone, 1,8-cineole (eucalyptol), (−)-carvone, 4-terpineol, thymol, methyl salicylate and L-mentyl methyl ether.

Use can be made of menthol in pure form (natural or synthetic) and/or as a component of natural oils and/or menthol-containing fractions of natural oils, especially in the form of essential (that is obtained by steam distillation) oils of certain Mentha species, in particular from Mentha arvensis (corn mint) and Mentha piperita (peppermint), these include Mentha piperita oils having regional designations of origin of special areas of cultivation such as Willamette, Yakima and Madras, and also oils of the type of the abovementioned names.

Use can be made of (−)-carvone in pure form (natural or synthetic) and/or as a component of natural oils and/or menthol-containing fractions of natural oils, especially in the form of essential (that is obtained by steam distillation) oils of certain Mentha species, in particular from Mentha cardiaca or Mentha spicata.

Use can be made of anethole as cis- or trans-anethole or in the form of mixtures of the isomers. Use can be made of anethole here in pure form (natural or synthetic) and/or as a component of natural oils and/or anethole-containing fractions of natural oils, in particular in the form of anise oil, star anise oil or fennel oil or anethole-containing fractions thereof.

Eucalyptol can be used in pure form (natural or synthetic) and/or as a component of natural oils and/or eucalyptol-containing fractions of natural oils, for example in the form of laurel (bay leaf) oil, but preferably eucalyptus oils from Eucalyptus fruticetorum and/or Eucalyptus globulus and/or Eucalyptol-containing fractions thereof.

Under some circumstances, instead of, or in addition to, the refreshing action, a cooling action can also be desired.

Preferred cooling active compounds used for this purpose are menthone glycerol acetal (trade name: Frescolat®MGA, Symrise GmbH & Co KG, Holzminden, Germany), menthyl lactate (trade name: Frescolat®ML Symrise GmbH & Co KG, Holzminden, Germany; preferably menthyl lactate is 1-menthyl lactate, in particular 1-menthyl 1-lactate), substituted menthyl-3-carboxamides (e.g. menthyl-3-carboxylic acid N-ethylamide, also known as WS-3), 2-isopropyl-N-2,3-trimethylbutanamide (also known as WS-23), substituted cyclohexanecarboxamides, 3-menthoxypropane-1,2-diol, 2-hydroxyethylmenthyl carbonate, 2-hydroxypropylmenthyl carbonate, N-acetylglycine menthyl ester, isopulegol, menthyl hydroxycarboxylic esters (e.g. menthyl 3-hydroxybutyrate), monomenthyl succinate, 2-mercaptocyclodecanone, menthyl-2-pyrrolidin-5-onecarboxylate, 2,3-dihydroxy-p-menthane, 3,3,5-trimethylcyclohexanone glycerol ketal, 3-menthyl 3,6-di- and -trioxaalkanoate, 3-menthyl methoxyacetate, icilin.

Particularly preferred cooling compounds are: menthone glycerol acetal, menthyl lactate (preferably 1-menthyl lactate, in particular 1-menthyl-lactate), substituted menthyl-3-carboxamides (e.g. menthyl-3-carboxylic acid N-ethylamide), 2-isopropyl-N-2,3-trimethylbutanamide, 3-menthoxypropane-1,2-diol, 2-hydroxyethyl menthyl carbonate, 2-hydroxypropyl menthyl carbonate, isopulegol and monomenthyl succinate.]

Preferably, the sensorially active compositions (II) used for the flavoring contain at least one refreshing compound and at least one cooling compound of the above-mentioned type. A preferred mixture of aroma substances therefore contains 1-menthol and at least one of the above-mentioned cooling substances.

It is likewise preferred to arrange the sensorially active compositions (II) in a manner such that, instead of, or in addition to a cooling and refreshing action, they also have a herbal, minty, cinnamon-like, clove-like, eucalyptus, wintergreen and/or fruity character.

Minty particularly includes peppermint and spearmint.

The above-mentioned aroma substances can be used in the sensorially active composition (II) individually or in any desired mixtures among one another.

Particularly preferably, the sensorially active composition (II) contains at least 3, most preferably at least 5, of the above-mentioned aroma substances.

Optically active aroma substances can be used in enantiomerically pure form, or as any desired mixtures of the two enantiomers. The same applies to (E)/(Z)-isomers and diastereomers.

The sensorially active compositions (II) can contain, as taste substances, for example, sugar replacers such as, mannitol, sorbitol and sorbitol syrup, isomalt (e.g. Palatinit®), maltitol and maltitol syrup, lactitol, xylitol, erythritol, leucrose, arabinol, arabitol, adonitol, alditol, ducitol, iditol, or else fructooligosaccharides (e.g. Raftilose®), oligofructose or polydextrose.

Typical sweeteners such as saccharin (if appropriate as Na, K or Ca salt), aspartame (e.g. NutraSweet®), cyclamate (if appropriate as Na or Ca salt), acesulfame-K (e.g. Sunett®), thaumatin, neohesperidin dihydrochalcone, stevioside, rebaudioside A, glycyrrhizin, ultrasweet, osladin, brazzein, miraculin, pentadin, phyllodulcin, dihydrochalcones, arylureas, trisubstituted guanidines, glycyrrhizin, superaspartame, suosan, sucralose (trichlorogalactosucrose, TGS), alitame, monellin or Neotame® can also be used.

Substances which can likewise be present are pungent-tasting substances and/or substances stimulating the flow of saliva in the mouth and/or substances causing a feeling of heat and/or a prickling feeling on the skin or on the mucosae. Examples of such compounds are capsaicin, dihydrocapsaicin, gingerols, paradols, shogaols, piperin, carboxylic acid N-vanillylamides, in particular nonanoic acid N-vanillylamide, pellitorin and spilanthol, 2-nonenoamides, in particular 2-nonenoic acid N-isobutylamide, 2-nonenoic acid N-4-hydroxy-3-methoxyphenylamide, alkylethers of 4-hydroxy-3-methoxybenzyl alcohol, in particular 4-hydroxy-3-methoxybenzyl n-butyl ether, alkylethers of 4-acyloxy-3-methoxybenzyl alcohol, in particular 4-acetyloxy-3-methoxybenzyl n-butyl ether and 4-acetyloxy-3-methoxybenzyl n-hexyl ether, alkylethers of 3-hydroxy-4-methoxybenzyl alcohol, alkylethers of 3,4-dimethoxybenzyl alcohol, alkylethers of 3-ethoxy-4-hydroxybenzyl alcohol, alkylethers of 3,4-methylenedioxybenzyl alcohol, (4-hydroxy-3-methoxyphenyl)acetamides, in particular (4-hydroxy-3-methoxyphenyl)acetic acid N-n-octylamide, vanillomandelic acid alkylamides, ferulic acid phenethylamides, nicotinaldehyde, methyl nicotinate, propyl nicotinate, 2-butoxyethyl nicotinate, benzyl nicotinate, 1-acetoxychavicol, polygodial and isodrimeninol, further preferably cis- and/or trans-pellitorin according to WO 2004/000787 and WO 2004/043906, alkenecarboxylic acid N-alkylamides according to WO 2005/044778, mandelic acid alkylamides according to WO 03/106404 or alkyloxyalkanoamides according to WO 2006/003210.

Preferred pungent-tasting natural extracts and/or natural extracts causing a feeling of heat and/or a prickling feeling on the skin or on the mucosae are those from paprika, pepper (for example, capsicum extract), chili pepper, ginger root, Aframomum melgueta, Spilanthes acmella, Kaempferia galanga or Alpinia galanga.

In addition, substances can be present to mask one or more unpleasant taste impressions, in particular a bitter, astringent and/or metallic taste impression or aftertaste. Examples which may be mentioned are lactisol [2O-(4-methoxyphenyl)lactic acid] (cf. U.S. Pat. No. 5,045,336), 2,4-dihydroxybenzoic acid potassium salt (cf. U.S. Pat. No. 5,643,941), ginger extracts (cf. GB 2,380,936), neohesperidin dihydrochalcone (cf. Manufacturing Chemist 2000, July issue, pp. 16-17), flavones (2-phenylchrom-2-en-4-ones) (cf. U.S. Pat. No. 5.580,545), certain nucleotides, such as cytidine 5′-monophosphates (CMP) (cf. US 2002/0177576), sodium salts such as sodium chloride, sodium citrate, sodium acetate, and sodium lactate (cf. Nature, 1997, volume 387, page 563), lipoproteins of β-lactoglobulin and phosphatidic acid (cf. EP-A 635 218), neodiosmin [5,7-dihydroxy-2-(4-methoxy-3-hydroxyphenyl)-7-O-neohesperidosyl-chrom-2-en-4-one] (cf. U.S. Pat. No. 4,154,862), preferably hydroxyflavanones according to EP 1 258 200, in turn preferably 2-(4-hydroxyphenyl)-5,7-dihydroxy-chroman-4-one (naringenin), 2-(3,4-dihydroxyphenyl)-5,7-dihydroxychroman-4-one (eriodictyol), 2-(3,4-dihydroxyphenyl)-5-hydroxy-7-methoxychroman-4-one (eriodictyol 7-methyl ether), 2-(3,4-dihydroxyphenyl)-7-hydroxy-5-methoxychroman-4-one (eriodictyol 5-methyl ether) and 2-(4-hydroxy-3-methoxyphenyl)-5,7-dihydroxychroman-4-one (homoeriodictyol), their (2S)- or (2R)-enantiomers or mixtures of same and also their monovalent or polyvalent phenolate salts with Na⁺, K⁺, NH₄⁺, Ca²⁺, Mg²⁺ or Al³⁺ as counterions, or γ-aminobutyric acid (4-aminobutanoic acid, as neutral form (“internal salt”) and in the carboxylate or ammonium form) according to WO 2005/096841.

Substances which have bitter, astringent, sticky, dusty, dry, mealy, rancid or metallic taste are, for example: xanthine alkaloids xanthines (caffeine, theobromine, theophylline), alkaloids (quinine, brucine, nicotine), phenolic glycosides (e.g., salicin, arbutin), flavonoid glycosides (for example hesperidin, naringin), chalcones and chalcone glycosides, hydrolyzable tannins (gallic acid or elagic acid esters of carbohydrates, e.g., pentagalloyl glucose), non-hydrolyzable tannins (if appropriate galloylated catechins and epicatechins and oligomers thereof, e.g., proanthyocyanidins or procyanidins, thearubigenin), flavones (e.g., quercetin. taxifolin, myricetin), other polyphenols (γ-oryzanol, caffeic acid and esters thereof), terpenoid bitter substances (e.g., limonoids such as limonin or nomilin from citrus fruits, lupulones and humulones from hops, iridoids, secoiridoids), absinthin from wormwood, amarogentin from gentian, metallic salts (potassium chloride, sodium sulfate, magnesium sulfate), certain pharmaceutical active compounds (e.g., fluoroquinolone antibiotics, paracetamol, aspirin, beta-lactam antibiotics, ambroxol, propylthiouracil [PROP], guaifenesin), certain vitamins (for example, vitamin H, vitamins of the B series such as vitamin B1, B2, B6, B12, niacin, pantothenic acid), denatonium benzoate, sucralose octaacetate, potassium chloride, magnesium salts, iron salts, aluminum salts, zinc salts, urea, unsaturated fatty acids, in particular, unsaturated fatty acids in emulsions, amino acids (e.g., leucine, isoleucine, valine, tryptophan, proline, histidine, tyrosine, lysine and phenylalanine), peptides (in particular peptides having an amino'acid from the group leucine, isoleucine, valine, tryptophan, proline and phenylalanine at the N- or C-terminus).

Such a taste can also be possessed by sweeteners or sugar replacers such as aspartame, neotame, superaspartame, saccharin, sucralose, tagatose, monellin, steviosides, thaumatin, miraculin, glycerrhizin and derivatives thereof, cyclamate and the pharmaceutically acceptable salts of the above-mentioned compounds.

The above-mentioned aroma and taste substances of the sensorially active compositions (II) are, preferably before incorporation into the chewable foams, first incorporated into a matrix (carrier) suitable for foods and items consumed for pleasure, e.g., in the form of emulsions, liposomes, e.g., starting from phosphatidylcholine, microspheres, nanospheres, or else in capsules, granules or extrudates. Preferably, the matrix is chosen in each case in a manner such that the taste and/or aroma substances are released from the matrix in a delayed manner, so that a long-lasting action is achieved.

Preferred matrices are selected here from the following group: polysaccharides such as starch, starch derivatives, cellulose and cellulose derivatives (such as hydroxypropylcellulose), alginates, gellan gum, agar and carrageenan, natural fats, natural waxes such a beeswax, carnauba wax, proteins such as gelatin, complexing agents such as cyclodextrins or cyclodextrin derivatives, preferably beta-cyclodextrin.

The loading of the matrices with taste and/or aroma substances to be used according to the invention can vary depending on requirement and desired sensory profile. Customarily, the loading of taste and/or aroma substances is 1 to 60% by weight, preferably 5 to 40% by weight, based on the total weight of matrix (carrier) and taste and/or aroma substances.

The amounts of sensorially active compositions (II) given herein relate to the total mass of the taste and/or aroma substances used. These amounts also include any amounts of matrices or carrier materials for the taste and/or aroma substances present.

It has in addition proven to be advantageous to convert the taste and/or aroma substances of the sensorially active component, before incorporation into the chewable foams, into a spray-dried form. Preferred matrices are starches, degraded starches, chemically or physically modified starches, modified celluloses, gum arabic, ghatti gum, tragacanth, karaya gum, carrageenan, guar seed meal, carob bean meal, alginates (e.g., Na-alginate), pectin, inulin, xanthan gum or maltodextrins, individually or in any desired mixtures with one another.

Particularly preferred carriers for providing spray-dried taste and/or aroma substances are maltodextrins, and also mixtures of maltodextrins and gum arabic, in, each case maltodextrins having DE values in the range 15 to 20 being particularly advantageous.

The degree of decomposition of the starch is measured by the:characteristic “dextrose equivalent” (DE), which can assume the limiting values 0 for the long-chain glucose polymer and 100 for pure glucose.

The encapsulation of taste and/or aroma substances by spray drying is known to those skilled in the art and described, for example, in U.S. Pat. No. 3,159,585, U.S. Pat. No. 3,971,852, U.S. Pat. No. 4,532,145 and U.S. Pat. No. 5,124,162. Spray-dried flavors are commercially available in many different flavor directions and particle sizes.

Suitable foam agents/stabilizers (III) include any of the commercially conventional foaming agents and foam stabilizers such as water-soluble fatty amides, sulfosuccinamides, hydrocarbon sulfonates, hydrocarbon sulfates or fatty acid salts, the lipophilic radical preferably containing 12 to 24 carbon atoms.

Preferred foam agents/stabilizers (III) are alkane sulfonates and alkane sulfates having 12 to 22 carbon atoms in the hydrocarbon radical, akylbenzo sulfonates and alkylbenzo sulfates having 14 to 24 carbon atoms in the hydrocarbon radical, and fatty amides and fatty salts having 12 to 24 carbon atoms and alkyl amine oxides.

The above-mentioned fatty amides are preferably fatty amides of mono- or di-(C2-3-alkanol)amines. Suitable fatty acid salts include, for example, alkali metal salts, amine salts, or unsubstituted ammonium salts.

Such fatty acid derivatives are typically based on fatty acids such as lauric acid, myristic acid, palmitic acid, oleic acid, stearic acid, ricinoleic acid, behenic acid or arachidic acid, coconut fatty acid, tallow fatty acid, soya fatty acid and hydrogenation products thereof.

Particularly preferred foam agents/stabilizers (III) are sodium lauryl sulfate, sodium lauryl ether sulfates, sulfosuccinamides, ammonium stearates, and also mixtures thereof.

Thickeners (IV) in the context of the present invention are compounds which enable the viscosity of the mixture of chewable foam-forming components I-V to be adjusted in a manner such that the generation and processing of the foam is promoted. Suitable thickeners are commercially conventional thickeners such as natural organic thickeners, e.g., dextrins and starch, organically modified natural substances, e.g., cellulose ethers and hydroxyethyl cellulose, organically fully synthetic substances, e.g., polyacrylic acids, polyvinylpyrrolidones, poly(meth)acrylic compounds and polyurethanes (associative thickeners), and also inorganic thickeners, e.g., benonites and silicic acids. Preferably organically fully synthetic thickeners are used. Acrylate thickeners which are diluted with water, if appropriate, before addition are particularly preferred. Preferred commercially conventional thickeners are, for example, Mirox® AM (BGB Stockholm GmbH, Krefeld, Germany), Walocel® MT 6000 PV (Wolff Cellulosics GmbH & Co KG, Walsrode, Germany), Rheolate® 255 (Elementies Specialities, Gent, Belgium), Collacral® VL (BASF AG, Ludwigshafen, Germany).

Cosmetic additives (V) in the context of the present invention include: preservatives, abrasives (polishing agents), antibacterial agents, anti-inflammatory agents, irritation-preventing agents, irritation-inhibiting agents, antimicrobial agents, antioxidants, astringents, antistatics, binders, (mineral) fillers, buffers, carrier materials, chelators (chelating agents), cleaning agents, care agents, surface-active substances, emulsifiers, enzymes, fibers, film-forming agents, fixatives, foam-forming agents, substances for preventing foam, foam boosters, gelling agents, gel-forming agents, moisturizers, moistening substances, moisture-retention substances, bleaching agents, brightening agents (for example, hydrogen peroxide), impregnating agents, friction-reducing agents, lubricants, clouding agents, plasticizing agents, opacifiers, glossing agents, silicones, mucosae-calming agents, mucosae-cleaning agents, mucosae-care agents, mucosae-healing agents, mucosae-protecting agents, stabilizers, suspension agents, vitamins, fatty oils, waxes, fats, phospholipids, saturated fatty acids, monounsaturated or polyunsaturated fatty acids, polyhydroxy fatty acids, liquefiers, dyestuffs, color-protecting agents, pigments, surfactants, silicone derivatives, polyols, organic solvents, silicic acids, calcium carbonate, calcium hydrogen phosphate, aluminum oxide, fluorides, salts of zinc, tin, potassium, sodium and strontium, pyrophosphates, and hydroxyapatites.

Antioxidants or substances having antioxidant activity of component V suitable for use in the chewable foams of the present invention include tocopherols and derivatives thereof, tocotrienols, flavonoids, ascorbic acid and salts thereof, alpha-hydroxy acids (e.g., citric acid, lactic acid, malic acid, tartaric acid) and Na, K and Ca salts thereof, components, extracts and fractions thereof isolated from plants, e.g., from tea, green tea, algae, grape pips, wheat germ, rosemary, oregano; flavonoids, quercetin, and phenolic benzylamines. In addition, suitable antioxidants are propyl gallate, octyl gallate, dodecyl gallate, butylated hydroxyanisole (BHA, E320), butylated hydroxytoluene (BHT, 2,6-di-tert-butyl-4-methylphenol, E321), lecithins, mono- and diglycerides of edible fatty acids esterified with citric acid, orthophosphates and Na, K and Ca salts of monophosphoric acid, and ascorbyl palmitate.

Dyes and pigments of component (V) suitable for use in the chewable foams of the present invention include: lactoflavin (riboflavin), beta-carotene, riboflavin-5′-phosphate, alpha-carotene, gamma-carotene, cantaxanthin, erythrosin, curcumin, quinolin yellow, yellow orange S. tartrazine, bixin, norbixin (annatto, orlean), capsanthin, capsorubin, lycopene, beta-apo-8′-carotenal, beta-apo-8′-carotenic acid ethylester, xanthophylls (flavoxanthin, lutein, cryptoxanthin, rubixanthin, violaxanthin, rodoxanthin), fast carmine (carminic acid, cochenille), azorubin, cochenille red A (Ponceau 4 R), beet red, betanin, anthocyans, amaranth, patent blue V, indigotin I (indigo carmine), chlorophylls, copper compounds of chlorophylls, acid brilliant green BS (lissamine green), brilliant black BN, Carbo medicinalis vegetabilis, titanium dioxide, iron oxides and iron hydroxides, calcium carbonate, aluminum, silver, gold, rubin pigment BK (lithol rubin BK), methyl violet B, victoria blue R, victoria blue B, acilan brillant blue FFR ((brilliant wool blue FFR), naphthol green B, acilan fast green 10 G (alkali fast green 10 G), ceres yellow GRN, Sudan blue II, ultramarine, phthalocyanine blue, phthalocyanine green, fast acid violet R. Naturally produced extracts (e.g., paprika extract, black carrot extract, red cabbage extract) can also be used for coloring purposes. Good results have also been achieved with the following colors: Aluminum Lakes: FD & C Yellow 5 Lake, FD & C Blue 2 Lake, FD & C Blue 1 Lake, Tartrazine Lake, Quinoline Yellow Lake, FD & C Yellow 6 Lake, FD & C Red 40 Lake, Sunset Yellow Lake, Carmoisine Lake, Amaranth Lake, Ponceau 4R Lake, Erythrosyne Lake, Red 2G Lake, Allura Red Lake, Patent Blue V Lake, Indigo Carmine Lake, Brilliant Blue Lake, Brown HT Lake, Black PN Lake, Green S Lake and mixtures thereof.

Suitable (mineral) fillers of component (V) are for example: calcium carbonate, titanium dioxide, silicon dioxide, talc, aluminum oxide, dicalcium phosphate, tricalcium phosphate, magnesium hydroxide and mixtures thereof.

Antimicrobial active compounds for improving oral hygiene can be of hydrophilic, amphoteric or hydrophobic nature. Examples of such antimicrobial active compounds are: triclosan, chlorhexidine and salts thereof (e.g., acetate, gluconate or hydrochloride thereof), peroxides, phenols and salts thereof, domiphen bromide (phenododecinium bromide), bromochlorophen, Zn salts, chlorophylls, Cu salts, Cu gluconate, Cu chlorophyll, sodium lauryl sulfate, quaternary monoammonium salts such as cocoalkylbenzyldimethylammonium chloride and pyridinium salts such as cetylpyridinium chloride. In addition to individual active compounds, use can be made of mixtures of active compounds or natural extracts or fractions thereof containing active compounds, such as those obtainable from neem, barberry, fennel, green tea, marigold, camomile, rosemary, thyme, propolis or turmeric.

Preferred cosmetic additives (V) are emulsifiers (e.g., lecithins, diacylglycerols, gum arabic), stabilizers (e.g., carageenan, alginate), preservatives (e.g., benzoic acid, sorbic acid), antioxidants (e.g., tocopherol, ascorbic acid), chelators (e.g., citric acid), plant extracts, natural or synthetic dyes or color pigments (e.g., carotinoids, flavonoids, anthocyans, chlorophyll and derivatives thereof) and/or antimicrobial active compounds.

In a preferred embodiment, the sensorially active and cosmetic components (II and V) of the chewable foams are suitable for oral hygiene products or dental care agents. To achieve this aim, use is preferably made of abrasives (or polishing agents), such as silicic acids, calcium carbonates, calcium phosphates, aluminum oxides and/or hydroxyl apatites; surface-active substances such as sodium lauryl sulfate, sodium lauryl sarcosinate and/or cocamidopropylbetaine; moisture-retention agents such as glycerol and/or sorbitol; sweeteners such as saccharin; taste-correcting agents for unpleasant taste impressions, taste-correcting agents for generally not unpleasant taste impressions, taste-modulating substances such as inositol phosphate, nucleotides such as guanosine monophosphate, adenosine monophosphate or other substances such as sodium glutamate or 2-phenoxypropionic acid; carboxymethylcellulose; polyethylene glycols; carrageenan and/or Laponite® magnesium silicate; active compounds such as sodium fluoride, sodium monofluorophosphate, tin difluoride, quaternary ammonium fluorides, zinc citrate, zinc sulfate, tin pyrophosphate, tin dichloride, mixtures of different pyrophosphates, triclosan, cetylpyridinium chloride, aluminum lactate, potassium citrate, potassium nitrate; potassium chloride, strontium chloride, hydrogen peroxide and/or sodium bicarbonate.

In addition, for this purpose, use can be made of substances for combating or preventing plaque, tartar or caries, and also those substances for combating or preventing mouth odor as are described in U.S. Pat. No. 5,043,154. Those which may be mentioned by way of example are Zn salts, such as Zn citrate, Zn fluoride; Sn salts, such as Sn fluorides; Cu salts; fluorides, e.g. amine fluorides; alkali metal fluorides such as Na fluoride; alkaline earth metal fluorides; ammonium fluoride; phosphates; pyrophosphates; fluorophosphates such as Na monofluorophosphate, Al monofluorophosphate and Al difluorophosphate; alpha-ionone; geraniol; thymol; isomenthyl acetate; panthenol (provitamin B5); xylitol; allantoin; niacinamide.(vitamin B3); tocopheryl acetate (vitamin E actetate); and poloxamer.

Preferably, the chewable foams flavored according to the invention are free from cariogenic substances such as sucrose, glucose, lactose, hydrolyzed lactose, sorbose, arabinose, xylose, mannose, maltose, galactose, maltotriose and fructose.

In the inventive method, 80 to 99.5% by weight of the polyurethane dispersion (I), 0.1 to 30% by weight of component (II), 0 to 10% by weight of component (III), 0 to 10% by weight of component (IV), and 0 to 15% by weight of component (V) are used, the quantities being based on the corresponding anhydrous components (I) to (V) and the sums of the anhydrous individual components totalling 100% by weight.

Preference is given to 80 to 99.5% by weight of the polyurethane dispersion (I), 1 to 15% by weight of component (II), 0.1 to 10% by weight of component (III), 0.1 to 10% by weight of component (IV) and 0.1 to 15% by weight of component (V), the quantities being based on the corresponding anhydrous components (I) to (V), and the sum of the anhydrous individual components totalling 100% by weight.

The foam can be produced by input of air and/or the action of corresponding shearing energy (for example, mechanical agitation) or by conventional blowing agents. The input of air under the action of corresponding shearing energy is preferred.

The addition of taste substances and/or aroma substances can proceed before, during and/or after the foam production. The taste substances and/or aroma substances can, for example, also already be present in the polyurethane-polyurea dispersion (I). Division of the taste substances and/or aroma substances and addition of the foam components and/or the foam at different points of the production process is also possible. In the case of liquid taste substances and/or aroma substances, the addition preferably proceeds before foam production. Solid taste substances and/or aroma substances are preferably added after foam production, during or after application of the foam to a surface or to a mold. Alternatively, or in addition, the chewable foam can also be admixed with taste substances and/or aroma substances.

The foamed composition can be applied in the most varied manner to the most varied surfaces or to molds, such as, for example, pouring, blade-coating, rolling, spreading, injecting or spraying. Shaping via an extrusion method is possible.

Whereas the foamed material before drying has a preferred foam density of from 200 to 700 g/l, preferably from 300 to 600 g/l, the density of the resultant inventive chewable foam after drying is preferably from 50 to 600 g/l, more preferably from 100 to 500 g/l.

The cell structure is generally dried at a temperature between 25° C. and 150° C., preferably between 30° C. and 150° C., more preferably from 40° C. to 140° C. Drying can, proceed in a conventional dryer or in a circulation oven. It is also possible for drying to proceed in a microwave (HF) dryer or an infrared dryer.

The flavored chewable foams according to the invention, after the drying step, typically have a thickness of from 1 mm to 100 mm, preferably from 1 mm to 50 mm, more preferably 1 mm to 30 mm, when they are applied as a layer, e.g., to a separation paper.

The flavored chewable foams according to the invention can also be applied to the most varied substrates in a plurality of layers, for example to generate particularly high foam layers, or can be cast into molds.

In addition, the foamed compositions according to the invention can also be used in combination with other support materials such as, for example, textile supports, paper etc., for example by previous application (e.g. coating).

The flavored chewable foams according to the invention have excellent mechanical properties, in particular a high extensibility at high tensile strength. They return to their original shape after the chewing process, have the ability to clean the chewing surfaces and teeth sides, have a pleasant taste, refresh the oral cavity region (oral, throat and/or nasal cavity) and do not stick to floor coverings.

### EXAMPLES

Unless stated otherwise, all percentages are by weight.

The solids contents were determined as specified in DIN-EN ISO 3251.

NCO contents were, unless explicitly stated otherwise, determined volumetrically as specified in DIN-EN ISO 11909.

Substances and abbreviations used:
Diaminosulfonate: NH₂—CH₂CH₂—NH—CH₂CH₂—SO₃Na (45% in water)
Desmophen® C2200: polycarbonate polyol, OH number 56 mg of KOH/g, number-average molecular weight 2000 g/mol (Bayer MaterialScience AG, Leverkusen, Germany)
PolyTHF® 2000: polytetramethylene glycol polyol, OH number 56 mg of KOH/g, number-average molecular weight 2000 g/mol (BASF AG, Ludwigshafen, Germany)
PolyTHF® 1000: polytetramethylene glycol polyol, OH number 112 mg of KOH/g, number-average molecular weight 1000 g/mol (BASF AG, Ludwigshafen, Germany)
Polyether LB 25: (monofunctional polyether based on ethylene oxide/propylene oxide number-average molecular weight 2250 g/mol, OH number 25 mg of KOH/g (Bayer MaterialScience AG, Leverkusen, Germany)
Stokal® SR: succinamate-based foam aid, active compound content: approximately 34% (Bozzetto GmbH, Krefeld, Germany)
Mirox AM: aqueous acrylic acid copolymer dispersion (BGB Stockhausen GmbH, Krefeld, Germany)
Octosol SLS: aqueous sodium lauryl sulfate solution (Tiarco Chemical Europe GmbH, Nuremberg, Germany)
Octosol 845: sodium lauryl sulfate ether (Tiarco Chemical Europe GmbH, Nuremberg, Germany)

The mean particle sizes (the number-average is reported) of the PUR dispersions were determined using laser correlation spectroscopy (instrument: Malvern Zetasizer 1000, Malver Inst. Limited).

### Example 1

### PUR Dispersion (Component I)

761.3 g of Desmophen® C2200 polyol, 987.0 g of PolyTHF® 2000 polyol, 375.4 g of PolyTHF® 1000 polyol and 53.2 g of Polyether LB 25 were heated to 70° C. Subsequently, at 70° C., in the course of 5 min, a mixture of 237.0 g of hexamethylene diisocyanate and 313.2 g of isophorone diisocyanate was added to the polyol mixture and the resulting mixture was stirred under reflux until the theoretical NCO value was achieved. The finished prepolymer was dissolved with 4850 g of acetone at 50° C. and subsequently a solution of 1.8 g of, 25.1 g of ethylenediamine, 61.7 g of diaminosulfonate, 116.5 g of isophoronediamine and 1030 g of water was added in the course of 10 min. The post-stirring time was 10 min. Thereafter, the mixture was dispersed by addition of 1061 g of water. The solvent was removed by distillation in vacuum and a storage-stable dispersion having a solids content of 57% was obtained.

### Example 2

### Production of a Flavored Chewable Foam According to the Invention

1000 g of the dispersion (I) produced in Example I were mixed with 30 g of Octosol SLS (III), 20 g of Stokal SR (III), 20 g of Octosol 845 (III), 5 g of 5% strength ammonia solution and 15 g of Mirox AM (IV) and subsequently foamed by input of air using a hand-mixing apparatus. Then, with further stirring, 15 g of the aroma substance D-limonene (II) were added. The resultant foam density was 400 g/l. Thereafter the foamed paste was applied using a film-drawing apparatus consisting of two polished rollers which could be set to an exact spacing and in which case a separation paper had been placed in front of the rear roller. Using a blade thickness gauge, the spacing between paper and front roller was set. This spacing corresponded to the film thickness (wet) of the resultant coating which was selected in a manner such that a dry layer thickness >100 μm was achieved. Finally, the material was dried in a drying cabinet for 15 minutes at 80° C. After removal of the separation paper, the inventive chewable foam was obtained. The properties of this chewable foam are reported in Table 1.

TABLE 1
Properties of the chewable foam

| | 100% modulus | Extension | Tensile strength |
|---|---|---|---|
| Chewable foam of: | [MPa] | [%] | [MPa] |
| Example 2 | 0.8 | 710 | 5.2 |

The modulus at 100% extension was determined on films having a layer thickness > 100 μm.

### Example 3

### Production of a Flavored Chewable Foam According to the Invention

In a manner similar to that used in Example 2, 1000 g of the dispersion (I) produced in Example 1 were mixed with 30 g of Octosol SLS (III), 20 g of Stokal SR (III), 20 g of Octosol 845 (III), 5 g of 5% strength ammonia solution and 15 g of Mirox AM (IV) and subsequently foamed by introducing air using a hand-mixing apparatus. Then, with further stirring, 32 g of the aroma B (II) were added. The remainder of the procedure was performed in the same manner as in Example 2.

Aroma B in this case had the following composition (data in each case in % by weight):

0.1% neotame powder, 0.05% aspartame, 29.3% lemon oil, 29.3% orange oil, 2.97% sucralose, 2.28% triacetin, 5.4% diethyl tartrate, 12.1% peppermint oil yakima, 0.7% ethanol, 3.36% 2-hydroxyethyl rnenthyl carbonate, 3.0% 2-hydroxypropyl menthyl carbonate, 0.27% vanillin, 5.5% D-limonene, 5.67% L-menthyl acetate.

### Example 4

### Production of a Flavored Chewable Foam According to the Invention

In a manner similar to that used in Example 2, 1000 g of the dispersion (I) from Example 1 were mixed with 30 g of Octosol SLS (III); 20 g of Stokal SR (III), 20 g of Octosol 845 (III), 5 g of 5% strength ammonia solution and 15 g of Mirox AM (IV) and subsequently foamed by introducing air using a hand-mixing apparatus. Then, with further stirring, 65 g of Aroma P (II), a synthetic peppermint oil, were added. The remainder of the procedure was performed in the same manner as in Example 2.

Aroma P is a mixture of the following components (in parts by weight):

| | |
|---|---|
| Isobutyraldehyde | 0.5 |
| 3-Octanol | 0.5 |
| Dimethyl sulfide | 0.5 |
| Trans-2-flexenal | 1.0 |
| Cis-3-Hexenol | 1.0 |
| 4-Terpineol, natural | 1.0 |
| Isopulegol | 1.0 |
| Piperitone, natural from eucalyptus | 2.0 |
| Linalool | 3.0 |
| 8-Ocimenyl acetate, 10% in triacetin | 5.0 |
| Isoamyl alcohol | 10.0 |
| Isovaleraldehyde | 10.0 |
| Alpha-Pinene, natural | 25.0 |
| Beta-Pinene, natural | 25.0 |
| Neomenthol, racemic | 40.0 |
| Eucalyptol (1,8-cineole), natural | 50.0 |
| L-Menthyl acetate | 70.0 |
| L-Menthone | 220.0 |
| D-Isomenthone | 50.0 |
| L-Menthol | 484.5 |
| Total: | 1000.0 |

### Example 5

### Production of a Flavored Chewable Foam According to the Invention

In a manner similar to that used in Example 2, 1000 g of the dispersion (I) from Example I were mixed with 30 g of Octosol SLS (III), 20 g of Stokal SR (III), 20 g of Octosol 845 (III), 5 g of 5% strength ammonia solution, 15 g of Mirox AM (IV), 35 g of abrasive silicic acid (silicon dioxide) (V), 10 g of titanium dioxide (V) and 8 g of Cu chlorophyll extract (see, for example WO 00/48560; antimicrobial active compound and green dye) (V) and subsequently foamed by introducing air using a hand-mixing apparatus. Then, with further stirring, 45 g of Aroma X (II) were added. The remainder of the procedure was performed in the same manner as in Example 2.

Aroma X is a mixture of the following components (in parts by weight):

| | |
|---|---|
| Spearmint oil | 10.57 |
| Peppermint oil piperita | |
| (menthol content approximately 53% by weight) | 43.44 |
| Sucralose | 1.73 |
| Saccharin | 2.61 |
| Triethyl citrate | 0.10 |
| L-Menthol, natural | 15.59 |
| Triacetin (glycerol triacetate) | 0.07 |
| Benzyl alcohol | 0.07 |
| Capsicum extract 1 million Scoville | 0.07 |
| Ethanol | 5.03 |
| 2-Hydroxyethyl menthyl carbonate (cooling | |
| compound) | 3.19 |
| L-Menthyl methyl ether | 3.10 |
| Piperine | 0.01 |
| Cis-/trans-Pellitorin (20/80) (m/m) | 0.01 |
| Plant oil | 4.50 |
| D-Limonene | 2.16 |
| Anethole | 7.75 |

On chewing this flavored chewable foam according to the invention, a refreshing cooling and liberating feeling in the oral, throat and nasal cavity was perceived, at the same time the teeth were being cleaned.

### Example 6

### Production of a Flavored Chewable Foam According to the Invention

In a manner similar to that used in Example 2, 1000 g of the dispersion (I) from Example I were mixed with 30 g of Octosol SLS (III), 20 g of Stokal SR (III), 20 g of Octosol 845 (III), 5 g of 5% strength ammonia solution,. 15 g of Mirox AM (IV), and 30 g of a mixture of sodium fluoride (V), sodium monofluorophosphate (V), and cetylpyridinium chloride (V) and some red dye (V), and subsequently foamed by introducing air using a hand-mixing apparatus. Then, with further stirring, 28 g of Aroma Y (II), a cooling and refreshing cinnamon aroma, were added. The remainder of the procedure was performed in the same manner as in Example 2.

Aroma Y is a mixture of the following components (in parts by weight):

| | |
|---|---|
| Oil of clove buds | 4.09 |
| Cinnamon leaf oil | 0.64 |
| Peppermint oil Arvenisis | |
| (menthol content approximately 74% by weight) | 4.05 |
| L-Menthone | 2.00 |
| Sucralose | 2.73 |
| BHT (E321) | 0.09 |
| Cassia oil, redistilled, USP FCC | 8.55 |
| Capsicum extract, African | 0.14 |
| Cis-/trans-Pellitorin (20/80) (m/m) | 0.01 |
| Cooling compound menthyl-3-carboxylic acid- | |
| N-ethylamide (WS3) | 0.20 |
| Ethanol | 8.12 |
| Water | 0.50 |
| C₆-C₁₀ triglyceride fraction from coconut oil | 6.20 |
| Eugenol | 4.68 |
| Alpha-Methylcinnamaldehyde | 3.09 |
| Cinnamaldehyde | 52.41 |
| Heliopan | 2.50 |

On chewing this flavored chewable foam according to the invention, a refreshing, cooling and liberating feeling was perceived in the oral, throat and nasal cavity.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A flavored chewable foam based on polyurethane-polyureas and one or more sensorially active substance(s).

2. The flavored chewable foam of claim 1 having a 100% modulus of from 0.4 to 5.0 MPa, at a tensile strength of 1 to 55 MPa and an extensibility of from 300 to 1800%.

3. The flavored chewable foam of claim 1 in which the sensorially active substance(s) is/are
(a) volatile and perceptible orthonasally and/or retronasally or
(b) nonvolatile and perceptible by interaction with taste receptors in a human tongue.

4. The flavored chewable foam of claim 3 in which the sensorially active substance(s) comprise(s) at least one compound which generates a combined refreshing and cooling sensation.

5. The flavored chewable foam of claim 3 in which the sensorially active substance(s) (b) comprise(s) a sweetener and/or pungent-tasting substance which stimulates saliva flow and causes a heat and/or a tingling sensation on the skin or on mucosae.

6. The flavored chewable foam of claim 3 in which the sensorially active substances is/are incorporated into a matrix as carrier.

7. A process for producing the flavored chewable foam of claim 1 comprising:
a) foaming a polyurethane-polyurea dispersion (I),
b) drying the foamed dispersion, and
c) adding the a flavoring composition (II) comprising one or more sensorially active substance(s) during foaming of the dispersion, or during drying of the foamed dispersion or subsequent to drying of the foamed dispersion.

8. The process of claim 7 in which the polyurethane-polyurea dispersions (I) are obtainable by producing
A) isocyanate-functional prepolymers from
a 1) aliphatic or cycloaliphatic polyisocyanates,
a2) polymeric polyols having number-average molecular weights of 400 to 8000 g/mol and OH functionalities of 1.5 to 6,
a3) if appropriate hydroxy functional, ionic or potentially ionic and/or nonionic hydrophilizing agents,
B) their free NCO groups then being in whole or in part reacted with
b1) amino functional compounds having molecular weights of 32 to 400 g/mol and/or
b2) amino functional, ionic or potentially ionic hydrophilizing agents with chain extension and the prepolymers being dispersed in water before, during or after step B), if appropriate potentially ionic groups present being able to be converted into the ionic form by partial or complete reaction with a neutralizing agent.

9. The process of claim 8 in the prepolymer is produced from a polyisocyanate selected from the group consisting of 1,6-hexamethylene diisocyanate, isophorone diisocyanate, isomeric bis(4,4′-isocyanatocyclohexyl)-methanes and mixtures thereof and a polyol comprising at least 70% by weight of a polycarbonate polyol and/or a polytetramethylene glycol polyol.

10. The process of claim 7 in which the flavoring composition (II) comprises as sensorially active substance(s)
(a) one or more volatile substance(s) which is/are perceptible orthonasally and/or retronasally,
or
(b) one or more nonvolatile substance(s) perceptible by interaction with taste receptors of a human tongue.

11. The process of claim 10 in which the sensorially active substance(s) comprise(s) a combination of refreshing and cooling active compounds.

12. The process of claim 10 in which the sensorially active substance(s) comprise(s) a sweetener and/or a pungent-tasting substance which stimulates saliva flow and/or causes a feeling of heat and/or a prickling feeling on skin or on the mucosae.

13. The process of claim 10 in which the sensorially active substance(s) is/are incorporated into a matrix as carrier.

14. The process of claim 7 in which foam stabilizers (III), thickeners and cosmetic additives are used as further components.

15. A method for freshening an oral and throat cavity comprising chewing the flavored chewable foam of claim 1.
